(19) United States
(12) Patent Application Publication
(10) Pub. No.: US 2009/0093631 A1
(43) Pub. Date: Apr. 9, 2009

(54) Processes for the preparation of a linezolid intermediate, linezolid hydroxide

(76) Inventors: Ben-Zion Dolitzky, Petach Tikva (IL); Nurit Perlman, Kfar Saba (IL); Marina Kalujny, Ariel (IL)

Correspondence Address: KENYON & KENYON LLP, ONE BROADWAY, NEW YORK, NY, 10004, US

(21) Appl. No.: 12/231,769

(22) Filed: Sep. 5, 2008

Related U.S. Application Data
(60) Provisional application No. 60/967,892, filed on Sep. 6, 2007.
(60) Provisional application No. 60/993,506, filed on Sep. 11, 2007.

(51) Int. Cl. C07D 413/10

(52) U.S. Cl. 544/137

(57) ABSTRACT

Provided are methods for the enantiomeric purification of Linezolid hydroxide, comprising providing a solution or a slurry of Linezolid hydroxide and a solvent selected from alcohols and ketones and crystallizing Linezolid hydroxide from the solution or slurry to obtain Linezolid hydroxide with a low content of S isomer.

### CROSS-REFERENCE TO RELATED APPLICATIONS

This application claims the benefit of Provisional Application Ser. No. 60/967,892, filed Sep. 6, 2007, and Provisional Application Ser. No. 60/993,506, filed Sep. 11, 2008, each of which is incorporated herein by reference in its entirety.

### FIELD OF THE INVENTION

The present invention relates to improved methods of preparing a pure intermediate of Linezolid.

### BACKGROUND OF THE INVENTION

Linezolid [(S)—N-[[3-(3-Fluoro-4-morpholinyl)phenyl]-2-oxo-5-oxazolidinyl]methyl]acetamide] is an antimicrobial agent. Linezolid is an oxazolidinone, having the empirical formula C₁₆H₂₀FN₃O₄ and the following structure (I):

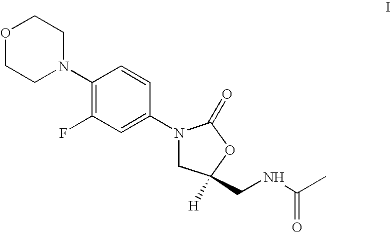

Linezolid is described in The Merck Index (13th edition, Monograph number: 05526, CAS Registry Number: 165800-03-3) as white crystals, with a melting point of 181.5-182.5° C. Linezolid, as well as a process for its preparation, is disclosed in U.S. Pat. No. 5,688,792 and International Patent Publication WO 95/07271.

This oxazolidinone is marketed as an injection, tablet, and oral suspension under the name ZYVOX®. It is mainly used to treat nosocomial pneumonia, skin and skin-structure infections, and vancomycin-resistant Enterococcus faecium infections. Linezolid hydroxide is used as an intermediate in the preparation of Linezolid. There are a number of methods for preparing Linezolid hydroxide described in the art. International Patent Application No. WO 97/37980 describes crystallization from a heptane and water mixture and subsequent removal of heptane. Disclosed therein is also a method of crystallization by dissolving in hot ethylacetate and addition of heptane. U.S. Pat. No. 5,688,792 describes crystallization from a mixture of ethyl acetate and hexane.

### SUMMARY

The present invention relates to a method for the purification of a Linezolid intermediate, Linezolid hydroxide, comprising providing a solution or a slurry of Linezolid hydroxide and a solvent selected from alcohols and ketones; and crystallizing to obtain Linezolid hydroxide having higher enantiomeric purity.

### DETAILED DESCRIPTION OF THE PREFERRED EMBODIMENTS

As used herein, “room temperature” refers to a temperature of about 20° C. to about 30° C., preferably about 25° C.

[(R)—N-[[3-(3-Fluoro-4-morpholinyl)phenyl]-2-oxo-5-oxazolidinyl]methanol] (Linezolid hydroxide) is an intermediate in the synthesis of Linezolid. It can be obtained commercially or prepared by any method known in the art. However, being an intermediate in the preparation of Linezolid, the enantiomeric purity of this intermediate affects the ultimate enantiomeric purity of Linezolid. Therefore it is important to utilize this intermediate in a level of high enantiomeric purity.

The present invention provides a method for the enantiomeric purification of Linezolid hydroxide (LNZ-OH), comprising:

a) providing a solution or a slurry of Linezolid hydroxide and a solvent selected from alcohols and ketones; and

b) crystallizing or precipitating Linezolid hydroxide from the solution or slurry to obtain Linezolid hydroxide with a content of S isomer that is lower than the content of S isomer in the Linezolid hydroxide of step a).

In one embodiment, the present invention relates to a method for the crystallization and purification of a Linezolid intermediate, Linezolid hydroxide, comprising providing a solution of Linezolid hydroxide and a solvent selected from alcohols and ketones; and crystallizing to obtain Linezolid hydroxide.

The solution of linezolid hydroxide in the organic solvent is prepared by dissolving Linezolid hydroxide in the organic solvent, for example by heating or by stirring for a sufficient period of time to dissolve the Linezolid hydroxide, preferably for about 10 minutes to about 1 hour, preferably about 15 minutes to about 30 minutes, more preferably about 15 minutes. Heating will depend on the solvent being used and is preferably from above room temperature to reflux, more preferably at reflux. The weight to volume ratio [g/mL] of linezolid hydroxide to the solvent is preferably from about 1:1 to about 1:15, more preferably from about 1:2 to about 1:9, and most preferably from about 1:2 to about 1:4.

Any alcohol or ketone may be used. A C₁-C₄ alcohol may be selected from the group consisting of methanol, isopropanol, ethanol, butanol, 2-butanol, propanol, and mixtures thereof. The ketone may be selected from the group consisting of C₃-C₆ ketones; preferably acetone.

The crystallization step is carried out by cooling to about −5° C. up to about 30° C. (for example, about 10° C. to about 30° C.), preferably about −5° C. to about 25° C., more preferably about 20° C. to about 25° C., and/or in some cases, also by adding anti-solvent such as water. Preferably, water is added to the heated solution, and, preferably, the solution is cooled to about 110° C. to about room temperature, preferably for about 4 hours to about 72 hours, more preferably for about 4 hours to about 20 hours, preferably about 16 hours. More preferably, water is added to the heated solution, and the solution is cooled to about room temperature for about 16 hours.

Once Linezolid hydroxide is obtained, it can be recovered by any means known in the art. Recovery, for example, may be by filtration and drying, preferably in vacuum. Appropriate time and temperature may be easily determined by the skilled artisan.

In another embodiment subsequent to obtaining Linezolid hydroxide, the process is repeated to further increase the content of the R isomer.

The resulting linezolid hydroxide is has a high enantiomeric purity. Preferably, the present process results in a reduction of the S isomer by at least 30%, preferably by at least 45%, and most preferably by at least 60%. Preferably, the resulting linezolid hydroxide has more than 99.4%, more preferably more than 99.6% and most preferably more than 99.8% of the R-isomer. Preferably, the resulting linezolid hydroxide has less than 0.6%, preferably less than 0.4% and more preferably less than 0.2% of the S-isomer and most preferably less than 0.15% of the S-isomer. The percentage of each isomer can be measured as area HPLC, e.g., by using the HPLC methods described herein.

In another embodiment, the present invention relates to another method for purification of a Linezolid intermediate, Linezolid hydroxide, comprising providing a slurry of Linezolid hydroxide and an alcohol; and precipitating to obtain Linezolid hydroxide. The process comprises exposing Linezolid hydroxide to an alcohol for at least a sufficient period of time to obtain more pure Linezolid hydroxide. Preferably the linezolid hydroxide is in a slurry.

Preferably, the slurry is maintained at about room temperature for about 4 to about 24 hours, more preferably about 10 hours to about 20 hours, most preferably for about 16 hours. Optionally, water is added and the slurry is maintained at about 10° C. to about room temperature for about 4 hours to about 24 hours. More preferably, water is added to and the slurry is maintained at about room temperature for about 16 hours.

Once Linezolid hydroxide is obtained, it can be recovered by any means known in the art. Recovery, for example, may be by filtration and drying, preferably in vacuum. Appropriate time and temperature may be easily determined by the skilled artisan.

In another embodiment, subsequent to obtaining Linezolid hydroxide, the process is repeated to further increase the content of the R isomer.

The weight to volume ratio [g/mL] of linezolid hydroxide to solvent is preferably from about 1:1 to about 1:15, more preferably from about 1:2 to about 1:9, and most preferably from about 1:2 to about 1:4.

Any alcohol may be used. A C₁-C₄ alcohol may be selected from the group consisting of: methanol, isopropanol, ethanol, butanol, 2-butanol, propanol, and mixtures thereof.

The resulting linezolid hydroxide has a higher enantiomeric purity. Preferably, the present process results in a reduction of the S isomer by at least 30%, preferably by at least 45%, and most preferably by at least 60%. Preferably, the resulting linezolid hydroxide has more than 99.4%, more preferably 99.6% and most preferably more than 99.8% of the R-isomer. Preferably, the resulting linezolid hydroxide has less than 0.6%, preferably less than 0.4% and more preferably less than 0.2% of the S-isomer and most preferably less than 0.15% of the S-isomer. The percentage of each isomer can be measured as area HPLC, e.g., by using the HPLC methods described herein.

The resulting pure linezolid hydroxide can then be subsequently converted to Linezolid by any means known in the art. Linezolid produced can then be used in the preparation of a medicament.

### EXAMPLES

### Instrumentation

HPLC

| | |
|---|---|
| Column & Packing: | Chiralpak AD-H 4.6*150 mm DAIC 19324 |
| Eluent | 40 Hexane: 60 Ethanol: 0.1 |
| | Trifluoroacetic acid (TFA) |
| Flow Rate: | 1.5 mL/min. |
| Detector: | 254 nm |
| Sample volume: | 20 microliters |
| Column temperature: | 25° C. |
| Autosampler temperature | 25° C. |
| Diluent: | Ethanol |

Mobile phase composition and flow rate may be varied in order to achieve the required system suitability.

Sample Solution Preparation

Weigh accurately about 10 mg of Linezolid sample in a 20 ml volumetric flask.

Dissolve and dilute with diluent using sonicator.

Procedure:

Inject the sample solution into the chromatograph, continuing the chromatogram of sample up to 1.5 times from Linezolid (R) peak. Determine the area for Linezolid (S) peak in each solution using a suitable integrator of only two peaks Linezolid (R) and Linezolid (S).

Calculation

$$\% LNZ \text{-} OH ( S ) = \frac{Area LNZ \text{-} OH ( S ) in sample}{Total area} \times 100$$

Crystallization Using Alcohols

### Example 1

Linezolid hydroxide (5 g, 0.6% (S isomer)) was slurried in methanol (10 ml) and stirred at for 16 h at RT. The product was isolated by vacuum filtration and dried in a vacuum oven (20 mbar-70 mbar) at 55° C. for 72 hours to obtain 3.5 g (70% yield, 0.33% (S isomer)).

### Example 2

Linezolid hydroxide (5 g, 0.6% (S isomer)) was dissolved in methanol (15 ml) by heating to reflux and stirred at reflux temperature for 15 min. Then water (30 ml) was added and the solution was cooled to room temperature and stirred for 16 hours. The product was isolated by vacuum filtration and dried in a vacuum oven (20 mbar-70 mbar) at 55° C. for 72 hours to obtain 3.56 g (71% yield, 0.21% (S isomer)).

### Example 3

Linezolid hydroxide (5 g, 0.6% (S isomer)) was slurried in methanol (10 ml) and stirred at for 16 h at RT. Water (20 ml) was added and the mixture was stirred at RT for 6 hours. The product was isolated by vacuum filtration and dried in a vacuum oven (20 mbar-70 mbar) at 55° C. for 16 hours to obtain 3.5 g (86% yield, 0.27% (S isomer)).

### Example 4

Linezolid hydroxide (5 g, 0.67% (S isomer)) was dissolved in ethanol (15 ml) by heating to reflux and stirred at reflux temperature for 15 min. Then the solution was cooled to room temperature and stirred for 16 hours. The product was isolated by vacuum filtration and dried in a vacuum oven (20 mbar-70 mbar) at 55° C. for 22 hours to obtain 4.09 g (81.8% yield, (0.29% (S isomer)).

### Example 5

Linezolid hydroxide (5 g, 0.6% (S isomer)) was dissolved in ethanol (15 ml) by heating to reflux and stirred at reflux temperature for 15 min. Then water (60 ml) was added and the solution was cooled to room temperature and stirred for 16 hours. The product was isolated by vacuum filtration and dried in a vacuum oven (20 mbar-70 mbar) at 55° C. over night to obtain 4.05 g 81% yield, 0.33% (S isomer)).

### Example 6

Linezolid hydroxide (4 g, 0.33% (S isomer)) obtained from Example 5 was dissolved in ethanol (36 ml) by heating to reflux and stirred at reflux temperature for 15 min. Then water (15 ml) was added and the solution was cooled to room temperature and stirred for 72 hours. The product was isolated by vacuum filtration and dried in a vacuum oven (20 mbar-70 mbar) at 55° C. for 16 hours to obtain 2.26 g (56.5% yield, 0.15% (S isomer)).

### Example 7

Linezolid hydroxide (5 g, 0.6% (S isomer)) was dissolved in 2-butanol (10 ml) by heating to reflux and stirred at reflux temperature for 15 min. Then water (15 ml) was added and the solution was cooled to room temperature and stirred for 16 hours. The product was isolated by vacuum filtration and dried in a vacuum oven (20 mbar-70 mbar) at 55° C. for 24 hours to obtain 1.93 g (38% yield, 0.29% (S isomer)).

### Example 8

Linezolid hydroxide (5 g, 0.6% (S isomer)) was dissolved in 2-butanol (10 ml) by heating to reflux and stirred at reflux temperature for 15 min. Then water (20 ml) was added and the solution was cooled to room temperature and stirred for 16 hours. The product was isolated by vacuum filtration and dried in a vacuum oven (20 mbar-70 mbar) at 55° C. for 16 hours to obtain 3.27 g (65% yield, 0.37% (S isomer)).

Crystallization Using Ketones

### Example 9

Linezolid hydroxide (5 g, 0.6% (S isomer)) was dissolved in Acetone (15 ml) by heating to reflux and stirred at reflux temperature for 15 min. Then water (20 ml) was added and the solution was cooled to room temperature and stirred for 16 hours. The product was isolated by vacuum filtration and dried in a vacuum oven (20 mbar-70 mbar) at 55° C. for 16 hours to obtain 1.78 g (35% yield, 0.28% (S isomer)).

### Example 10

LNZ-OH (3.47 g, 0.35% (S isomer)) was dissolved in Acetone (17.3 ml) by heating to reflux and stirred at reflux temperature for 15 min. Then water (47.2 ml) was added and stirred for 30 min and the solution was cooled to room temperature and stirred for 72 hours. The product was isolated by vacuum filtration and dried in a vacuum oven (20 mbar-70 mbar) at 55° C. for 16 hours to obtain 2.26 g (65%, 0.13% (S isomer)).

### Example 11

Linezolid hydroxide (5 g, 0.6% (S isomer)) was dissolved in Acetone (15 ml) by heating to reflux and stirred at reflux temperature for 15 min. Then water (30 ml) was added and the solution was cooled to room temperature and stirred for 16 hours. The product was isolated by vacuum filtration and dried in a vacuum oven (20 mbar-70 mbar) at 55° C. for 72 hours to obtain 3.48 g (69% yield, 0.28% (S isomer)).

### Example 12

Linezolid hydroxide (5 g, 0.6% (S isomer)) was dissolved in Acetone (15 ml) by heating to reflux and stirred at reflux temperature for 15 min. Then water (60 ml) was added and the solution was cooled to room temperature and stirred for 16 hours. The product was isolated by vacuum filtration and dried in a vacuum oven (20 mbar-70 mbar) at 55° C. for 16 hours to obtain 3.57 g (71.4% yield, 0.35% (S isomer)).

### Example 13

Linezolid hydroxide (3.47 g, 0.35% (S isomer)) obtained from example 11 was dissolved in Acetone (17.3 ml) by heating to reflux and stirred at reflux temperature for 15 min. Then water (47.2 ml) was added and stirred for 30 min and the solution was cooled to room temperature and stirred for 72 hours. The product was isolated by vacuum filtration and dried in a vacuum oven (20 mbar-70 mbar) at 55° C. for 16 hours to obtain 2.26 g (65% yield, 0.13% (S isomer)).

### Comparative Examples

The following crystallization methods failed to crystallize or failed to increase enantiomeric purity.

### Example 13 in Table

LNZ-OH (10 g, 0.67% (S isomer)) was dissolved in acetonitrile (30 ml) by heating to 81° C. and filtered at this temp.

Then water (30 ml) was added and solution was allowed to cool to room temperature and stirred overnight.

After stirring overnight, there was no crystallization. Seeding was done and the solution was stirred overnight.

No crystallization occurred.

### Example 14 in Table

LNZ-OH (5 g, 0.67% (S isomer)) was dissolved in acetonitrile (15 ml) by heating to reflux and then methyl tert-butylether (10 ml) was added.

The solution was cooled to room temperature and stirred overnight.

The product was isolated by vacuum filtration and dried in a vacuum oven at 55° C. for 22 hours to obtain 3.19 g (63%, 0.65% (S isomer)).

### Example 15 in Table

LNZ-OH (5 g, 0.67% (S isomer)) was dissolved in THF (10 ml) by heating to reflux, and then methyl tert-butylether (10 ml) was added.

The solution was cooled to room temperature and stirred overnight.

The product was isolated by vacuum filtration and dried in a vacuum oven at 55° C. for 22 hours to obtain 3.85 g (77%, 0.65% (S isomer)).

| Ex- | | Volume | | | % |
|---|---|---|---|---|---|
| am- | | [mL/g | | | Linezolid- |
| ple | | linezolid- | Tempera- | | OH S- |
| # | Solvent | OH] | ture | Yield % | isomer |
| 13 | Acetonitrile/ | 3/3 | reflux | No | |
| | water | | | | |
| | | | | crystallization | |
| 14 | Acetonitrile/ | 3/3 | reflux | 63.8 | 0.65 |
| | MTBE | | | | |
| 15 | THF/MTBE | 2/2 | reflux | 77 | 0.65 |

What is claimed is:

1. Linezolid hydroxide having a content of R isomer of more than about 99.4% relative to S isomer.

2. The Linezolid hydroxide of claim 1 having a content of R isomer of more than about 99.6% relative to S isomer.

3. The Linezolid hydroxide of claim 1 having a content of R isomer of more than about 99.8% relative to S isomer.

4. A method for enantiomeric purification of Linezolid hydroxide, comprising:
a) providing a solution or a slurry of Linezolid hydroxide and a solvent selected from alcohols and ketones; and
b) crystallizing or precipitating Linezolid hydroxide from the solution or slurry to obtain Linezolid hydroxide with a content of S isomer that is lower than the content of S isomer in the Linezolid hydroxide of step a).

5. The method of claim 4 where step a) provides a solution of Linezolid hydroxide and a solvent selected from the group consisting of alcohols and ketones.

6. The method of claim 5 where the solution is prepared by dissolving Linezolid hydroxide in the solvent by heating or by stirring for a sufficient period of time to dissolve the Linezolid hydroxide.

7. The method of claim 6 where the sufficient period of time is about 10 minutes to about 1 hour.

8. The method of claim 6 where heating is carried out from about room temperature to reflux.

9. The method of claim 4 where the weight to volume ratio [g/mL] of linezolid hydroxide to the solvent is from about 1:1 to about 1:15, from about 1:2 to about 1:9, or from about 1:2 to about 1:4.

10. The method of claim 4 where the solvent is an alcohol.

11. The method of claim 10 where the alcohol is a C₁-C₄ alcohol.

12. The method of claim 11 where the C₁-C₄ alcohol is selected from the group consisting of methanol, isopropanol, ethanol, butanol, 2-butanol, propanol, and mixtures thereof.

13. The method of claim 4 where the solvent is a ketone.

14. The method of claim 13 where the ketone is a C₃-C₆ ketone.

15. The method of claim 14 where the ketone is acetone.

16. The method of claim 4 where the crystallizing of step b) is carried out by cooling to about −5° C. to about 30° C., about −5° C. to about 25° C., or about 20° C. to about 25° C.

17. The method of claim 4 where the crystallizing of step b) is carried out by adding an anti-solvent.

18. The method of claim 17 where the anti-solvent is water.

19. The method of claim 4 where step a) provides a slurry of Linezolid hydroxide and an alcohol.

20. The method of claim 19 where the slurry is maintained at about room temperature for about 4 to about 24 hours.

21. The method of claim 19 where water is added and the slurry is maintained at about 10° C. to about room temperature for about 4 hours to about 24 hours, about 10 hours to about 20 hours, or about 16 hours.

22. The method of claim 19 where the weight to volume ratio [g/mL] of linezolid hydroxide to solvent is from about 1:1 to about 1:15, from about 1:2 to about 1:9, or from about 1:2 to about 1:4.

23. The method of claim 19 where the alcohol is a C₁-C₄ alcohol.

24. The method of claim 23 where the C₁-C₄ alcohol is selected from the group consisting of: methanol, isopropanol, ethanol, butanol, 2-butanol, propanol, and mixtures thereof.

25. The method of claim 4 where the obtained crystalline Linezolid hydroxide has a content of R isomer of more than 99.4%.

26. The method of claim 4 where the obtained crystalline Linezolid hydroxide has a content of R isomer of more than 99.6%.

27. The method of claim 4 where the obtained crystalline Linezolid hydroxide has a content of R isomer of more than 99.8%.

28. The method of claim 4 further comprising converting the obtained Linezolid hydroxide to Linezolid.
